(19) United States
(12) Patent Application Publication
(10) Pub. No.: US 2007/0265511 A1
(43) Pub. Date: Nov. 15, 2007

(54) Diabetes Blood Glucose Test Site Cleaning Kit

(76) Inventor: Elissa L. Renouf, Highvale (AU)

Correspondence Address: HOFFMAN WASSON & GITLER, P.C;CRYSTAL CENTER 2, SUITE 522, 2461 SOUTH CLARK STREET, ARLINGTON, VA, 22202-3843, US

(21) Appl. No.: 11/660,700

(22) PCT Filed: Aug. 26, 2005
(86) PCT No.: PCT/AU05/01292

§ 371 Date: Jul. 6, 2007

(30) Foreign Application Priority Data
Aug. 26, 2004 (AU) 2004904865

(51) Int. Cl. A61B 5/00; A61M 1/00

(52) U.S. Cl. 600/319; 604/66

(57) ABSTRACT

A diabetes blood glucose test site cleaning kit comprising a container housing (1) a multiplicity of swabs wherein the swabs are layered within the container to facilitate one by one removal and wherein the swabs are moistened with an aqueous cleansing solution, the container (1) being dimensioned such that the cleaning kit readily fits into a blood glucose meter wallet.

The present invention relates to the cleaning of test sites for blood glucose monitoring of diabetics. In particular, the present invention relates to a diabetes blood glucose test site cleaning kit.

People with diabetes need to monitor their blood glucose levels regularly in order to manage their diabetes and ensure their blood glucose levels remain within normal levels. The identification of low blood glucose levels, such as less than 5 mmol/L in children and 4 mmol/L in adults, is used to predict the risk of hypoglycaemia. Hypoglycaemia is often associated with a number of symptoms such as weakness, trembling or shaking, light headedness, dizziness, tearful crying, hunger, sweating, headaches, lack of concentration, behaviour changes, irritability and numbness around the lips and fingers. It is important that hypoglycaemia is treated immediately to stop the blood glucose levels from dropping lower. If hypoglycaemia is not treated immediately then the blood glucose levels may continue to drop and result in a loss of coordination, slurred speech, confusion or a loss of consciousness and fitting. This can be a life threatening condition.

The testing of blood sugar levels can also indicate higher than normal levels of blood glucose which can indicate the need to administer insulin in order to return the blood sugar levels to within the normal range.

Blood sugar levels are tested using a blood glucose meter, a lancet and test strips. Typically the finger is used as a test site for testing blood sugar levels and the finger is pricked with the lancet to obtain a very small drop of blood. The blood is applied to a test strip which is subsequently placed into the blood glucose meter and the blood sugar levels are displayed. In many situations, it is readily possible for the test site to be cleaned by washing the hands prior to the administration of the blood glucose level test. However, often the person with diabetes is not readily able to wash their hands prior to the administration of the test, such as when they are out. Surveys have shown that more than 40% of diabetics do not clean the test site before testing for blood glucose levels. As a result the possibility exists that the blood glucose level test may be artificially inflated by residual sugars that may contaminate the test site. This is particularly the case with children who notoriously seem to attract and collect contaminants on their hands as a result of tactile play with foods and the like. Alcohol wipes are generally not recommended for frequent use due to the tendency due dry out the skin. It has also been reported that alcohol wipes may effect the results of the test.

This can be particularly problematic as the artificial inflation of a blood glucose test result may lead to the unnecessary administration of insulin. Where the real blood glucose level is normal or even below normal but the test result indicates a high blood glucose level due to the contamination of the test site the administration of insulin may induce hypoglycaemia or worse. Whilst various wipes and other cleaning products are available commercially, a person suffering from diabetes often needs to carry a blood glucose meter wallet which may include a blood glucose meter, a lancet, test strips, an insulin pen or other insulin administration system and it is often not practicable to fit or carry commercial cleaning wipes or the like within the wallet. As a result the likelihood of falsely inflated blood glucose readings is increased.

We have now found a diabetes blood glucose test site cleaning kit which overcomes or ameliorates some of the above-mentioned problems or at least provides the user with a useful or commercial choice. According to the present invention we have found a diabetes blood glucose test site cleaning kit comprising a container housing a multiplicity of swabs wherein the swabs are layered within the container to facilitate one by one removal and wherein the swabs are moistened with an aqueous cleansing solution, the container being dimensioned such that the cleaning kit readily fits into a blood glucose meter wallet.

Blood glucose meter wallets are small, portable wallets used to contain the equipment essential for the management of diabetes in an insulin and non-insulin dependant diabetic person. Typically a blood glucose meter wallet will contain a blood glucose metre, a lancet and a number of test strips (typically up to 50) that can be used to determine the persons blood glucose level. The blood glucose meter wallet may also contain insulin which may be in the form of a predrawn syringe, an insulin pen or insulin and an undrawn syringe. In order to keep the blood glucose meter wallets relatively small and manageable, the wallets tend to be tightly packed and have little unused space. Typically, a blood glucose metre wallet will be about 150 mm by 200 mm in length and width and between about 20 and 40 mm in thickness. Blood glucose metre wallets are becoming increasingly small and may be as small as 100 mm by 100 mm in length and width.

The container for use in the present invention is dimensioned such that it can be fitted into such an blood glucose meter wallet and accordingly, it is preferred that the container have a foot print of less than the size of a credit card (about 55 mm×about 90 mm). The container more preferably has a footprint of about 54 mm×27 mm or about 54 mm×40 mm. The thickness of the container may be any convenient thickness to fit within the blood glucose meter wallet. We have found that a preferred thickness is in the range of from 10 to 20 mm which enables a useful number of swabs to be contained within the container whilst keeping the container size to a minimum. Preferably the container will house a number of swabs equivalent to the number of test strips carried in the wallet. Typically, this may be up to 50 swabs. Each swab may be of a size suitable for incorporation in the container and it is preferred that the swabs are about 95 mm×35 mm or 40 mm×40 mm. In one embodiment of the present invention the diabetes blood glucose test site cleaning kit may be sold with, or as part of a package of, test strips. Alternatively refills for the cleaning kit may be sold with test strips.

Refills for the container may be provided in a variety of packaging configurations that include simple sealed bags and sealed cartridges that are dimensioned to be accommodated by the container. In one embodiment the cartridge is formed from a soft packaging with a rigid rim for locating the packaging in the container.

In one configuration of the present invention, the container may have a retaining member that holds a refill in the container during use and that can be removed for replacement of the refill. Such a retaining member may have a centrally disposed slot through which the swabs may be removed. Alternatively, the retaining member may have an enlarged aperture through which the swabs may be removed from a refill that has a slot therein. It is preferable that the swabs are removed through a narrow slot so that loss of cleansing solution through evaporation is minimised.

In another configuration of the present invention, the container may include a sprung floor insert that elevates the swabs to the top of the container for ready access. A suitable sprung floor may include a support having integrally moulded, downwardly extending leaves that biases the support to a raised condition.

The container may be made of any convenient material, but for ease of manufacture, likeness and cost, it is preferred that the container be an injection moulded plastics container. It is preferred that the container have an integral hinge such that the swabs can be readily accessed by the user and the container resealed for subsequent storage and use.

The container may be configured for one or more recesses to house swabs. In one form, the container may have one recess for housing swabs moistened with an aqueous cleansing solution and another recess that may house swabs or wipes the are dry for use in drying the test site either before or after the blood test.

In one such configuration the recesses may be housed in side-to-side relationship and in another configuration the recesses may be housed in front-to-back relationship.

The swabs may be made of any convenient material and are preferably in the form of a sheet of a textile fabric, an absorbent plastic or a paper based material. The swabs may be simply layered in the container, one above each other such that the swabs can be removed one by one for cleansing the test site. Alternatively, the swabs may be interlayered, that is folded such that each swab extends partway into an adjacent layer leaving an end that may be readily accessed by the user for the removal of the next swab.

The swabs may be interlayered or interleaved in any convenient manner. We have found that by interleaving the swabs it is easy to withdraw one swab and position the next for withdrawal through a centrally disposed slot in the container. However, using a “z-fold” we are able to interlayer the swabs to get a somewhat greater packing density which is desirable, but not essential in the small packaging of the present invention.

The swabs are moistened with an aqueous cleansing solution. The aqueous cleansing solution may be any convenient cleansing solution and in one embodiment may simply be water. The cleansing solution for use in cleaning a blood glucose test site must be free of sugars such that the blood glucose level determined from the test is not affected by the cleansing solution. Thus the cleansing solution should be free of sugars or other substances that may effect the results of the blood glucose test. The cleansing solution may include suitable surfactants for assisting in the removal of sugars and other contaminants from the test site. The cleansing solution may also contain preservatives such as antibacterial agents to provide long life to the blood glucose test site cleansing kit.

The swabs may be used not only for cleaning the test site but may also be used for cleaning up excess blood that results from lancing the test site to obtain the blood sample. The cleaning of the test site after the blood glucose test is complete is desirable for hygienic reasons so that the risk of transmission of blood born diseases is minimised. The diabetes blood glucose test site cleaning kit of the present invention makes this post testing cleaning of the test site more convenient and more likely to be consistently practiced by the user.

It will be appreciated that the diabetes blood glucose test site cleansing kit of the present invention may be used to display selected information about site preparation for administering a blood glucose test or other aspects of diabetes management.

The diabetes blood glucose test site cleaning kit may include a fastener on the container for fixing the container to the wallet. Suitable fasteners may be of the hook and loop type or a mechanical or magnetic clasp such that the kit can be readily attached to and removed from the insulin wallet.

The present invention will now be described with reference to the accompanying drawings which have been included for illustrative purposes and do not serve to limit the scope of the invention disclosed.

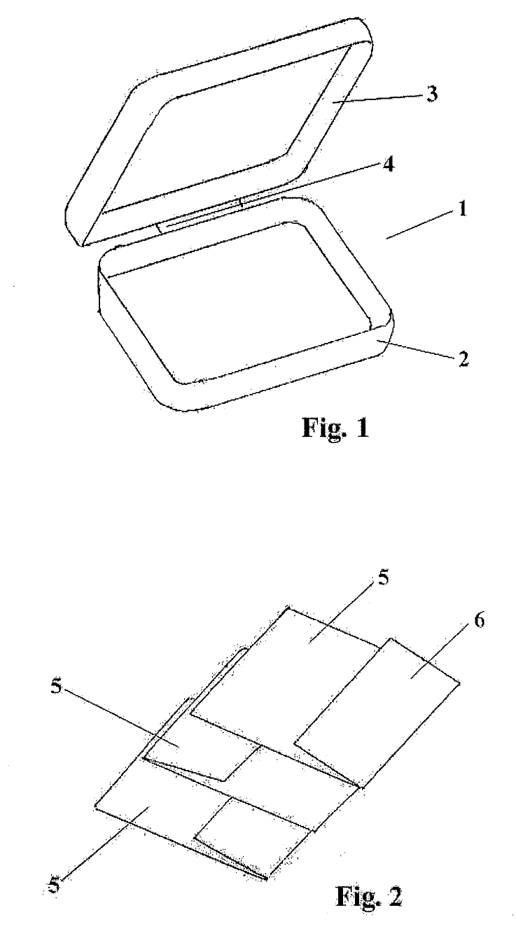

FIG. 1 shows a perspective view of a container for a diabetes blood glucose test site cleaning kit according to one embodiment of the present invention;

FIG. 2 shows folded swabs layered for incorporation into the container shown in FIG. 1;

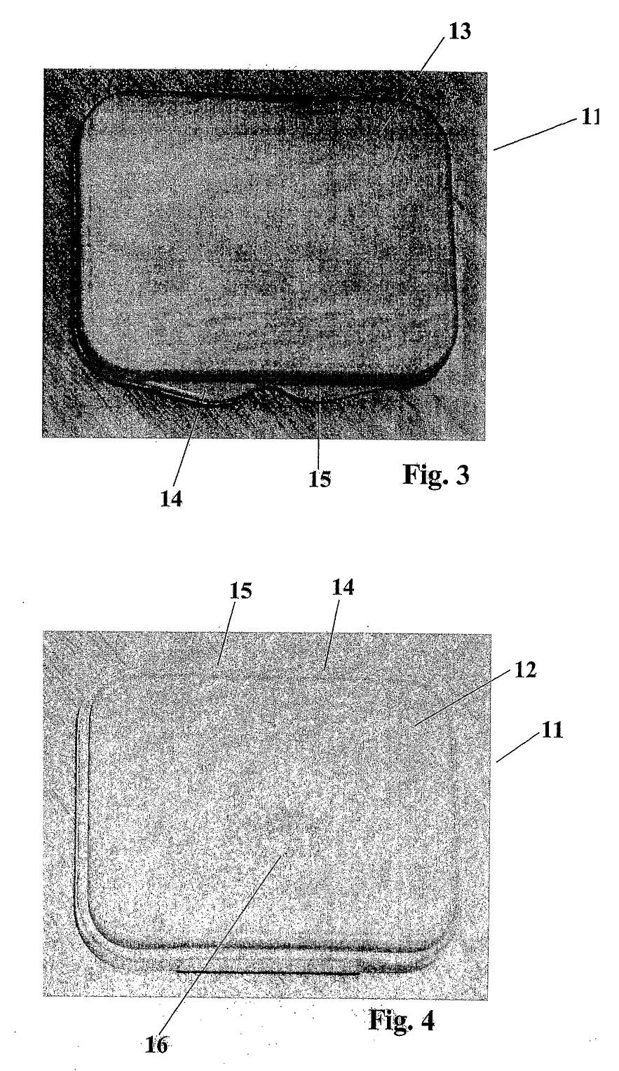

FIG. 3 shows a top view of a container according to a further embodiment of the present invention;

FIG. 4 shows a bottom view of a container shown in FIG. 3;

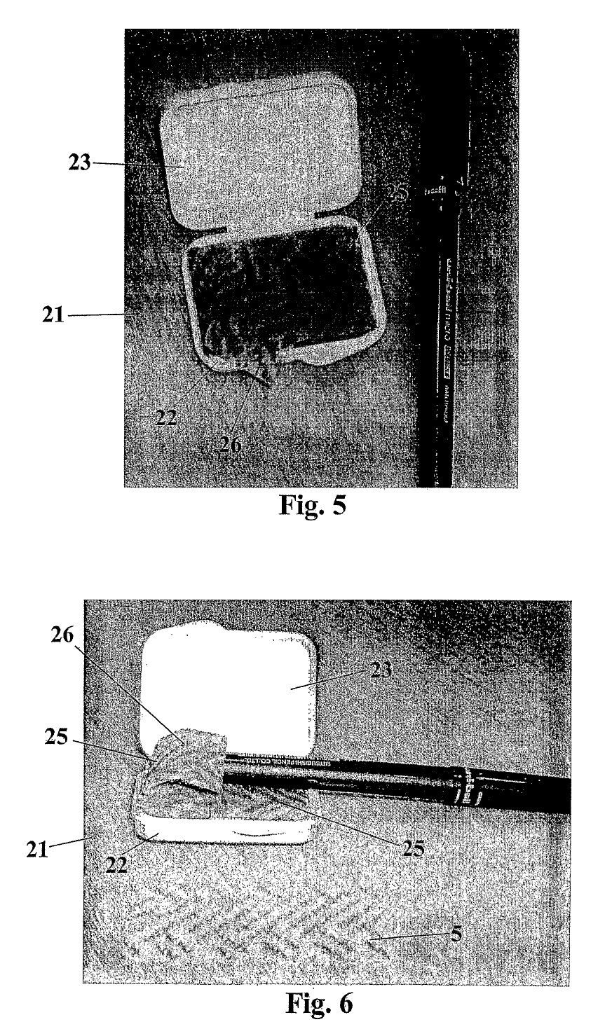

FIG. 5 shows a container according to another embodiment of the present invention in an open condition;

FIG. 6 shows the container of FIG. 5 with folded swabs layered in the container, as well as a swab in an unfolded form;

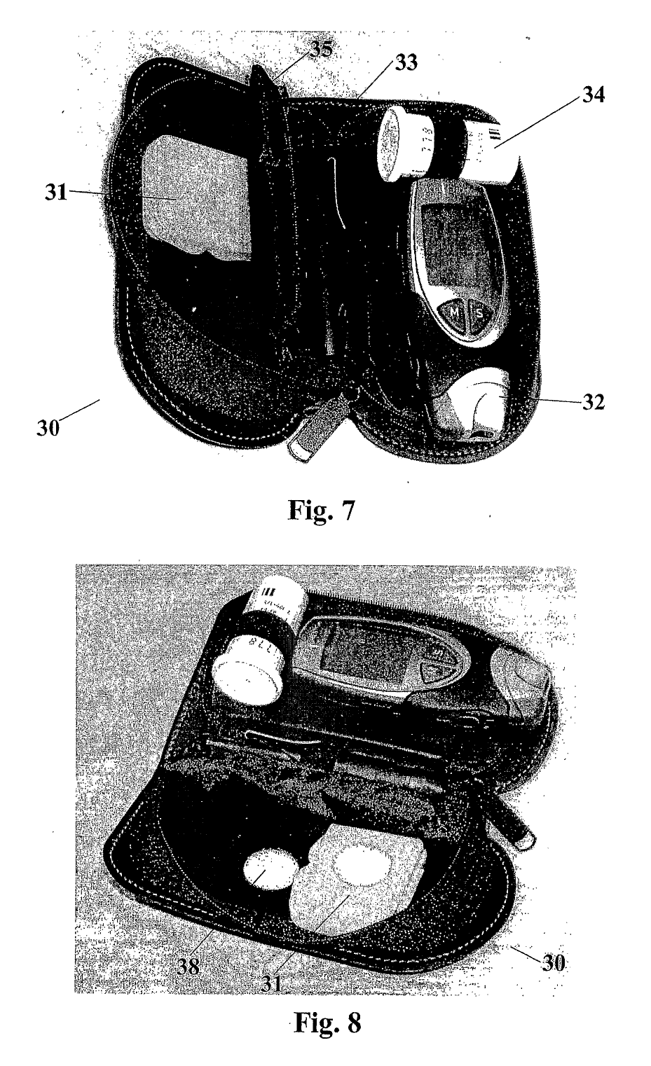

FIG. 7 shows a blood glucose test kit incorporating a diabetes blood glucose test site cleaning kit according to another embodiment of the present invention;

FIG. 8 shows the blood glucose test kit shown in FIG. 8;

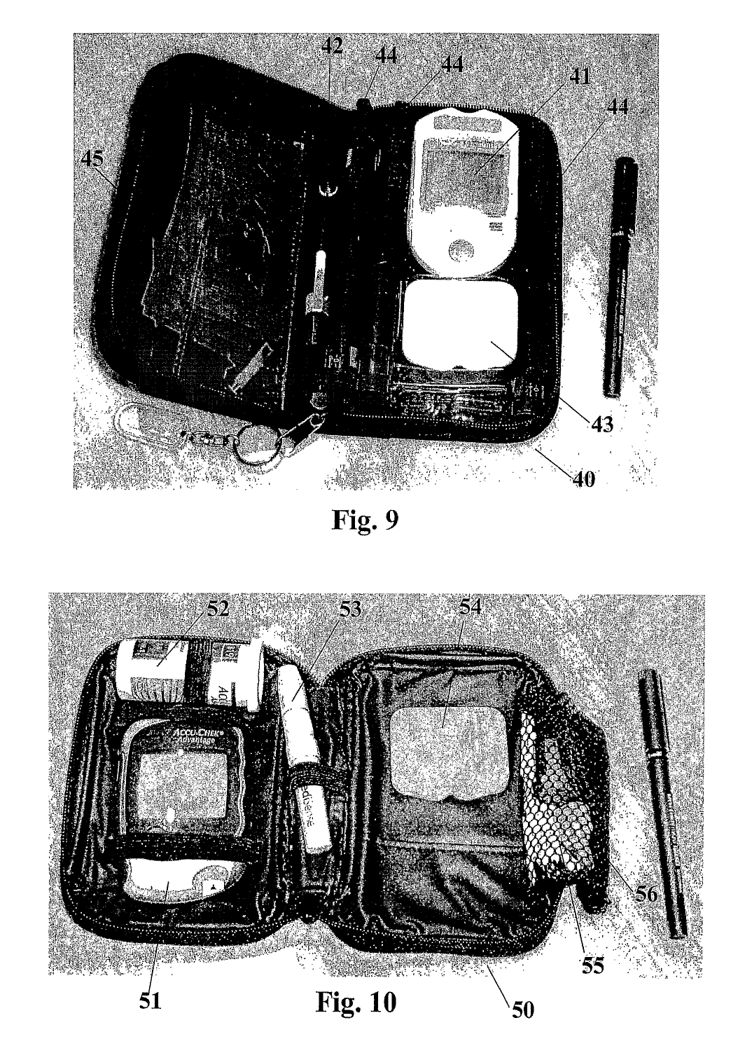

FIG. 9 shows a blood glucose test kit according to a further embodiment of the present invention; and

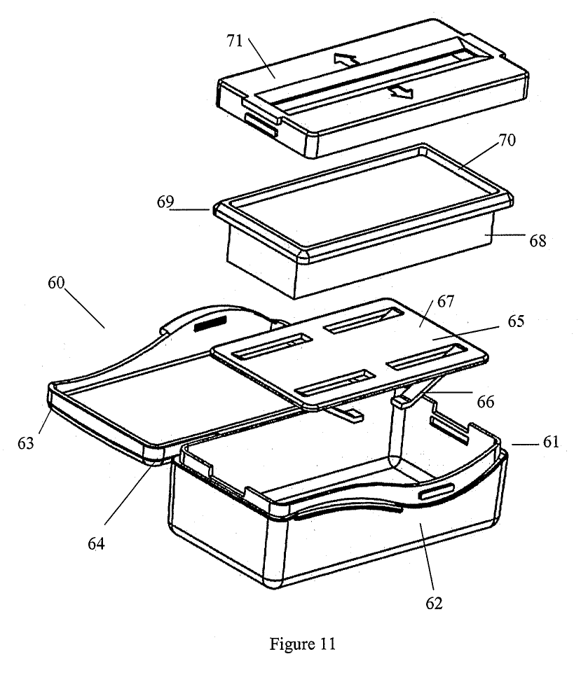

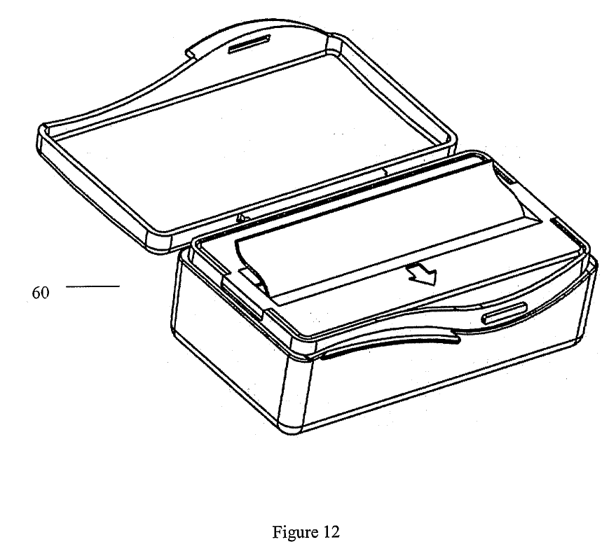

FIG. 10 shows a blood glucose test kit according to yet another embodiment of the present invention.

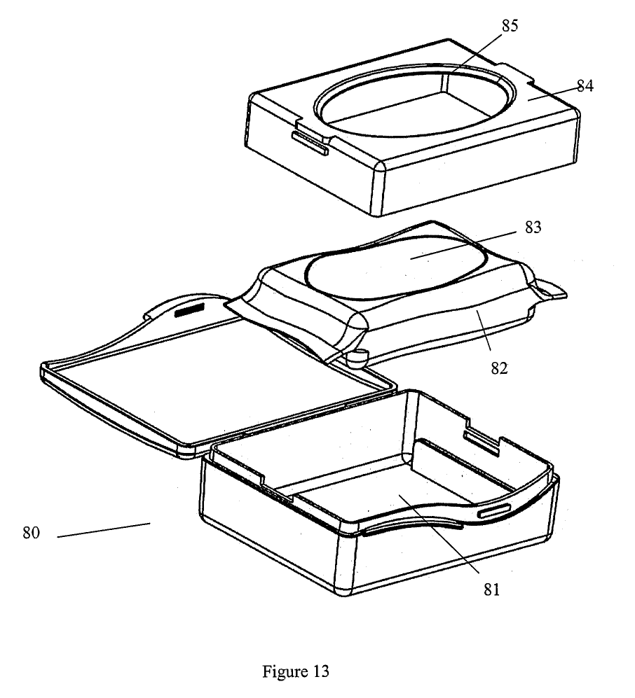

FIG. 11 shows an exploded view of a blood glucose test site cleaning kit according to one embodiment of the present invention.

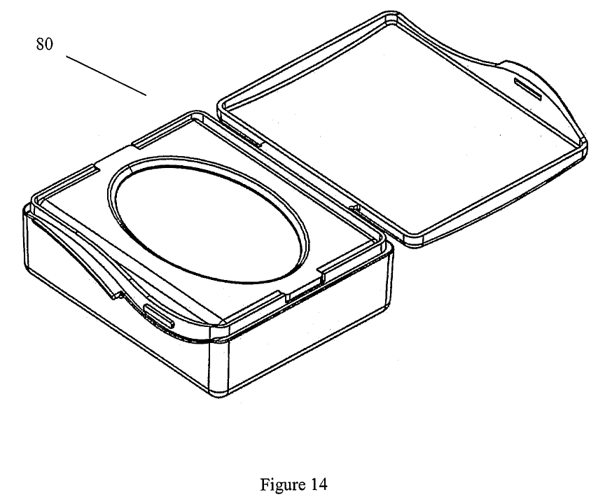

FIG. 12 shows an assembled view of the blood glucose test site cleaning kit shown in FIG. 11.

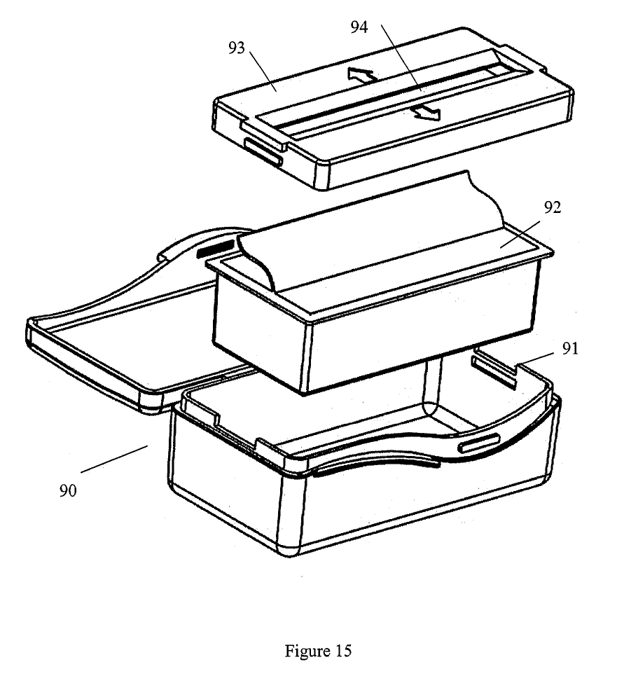

FIG. 13 shows an exploded view of a blood glucose test site cleaning kit according to another embodiment of the present invention.

FIG. 14 shows an assembled view of the blood glucose test site cleaning kit shown in FIG. 13.

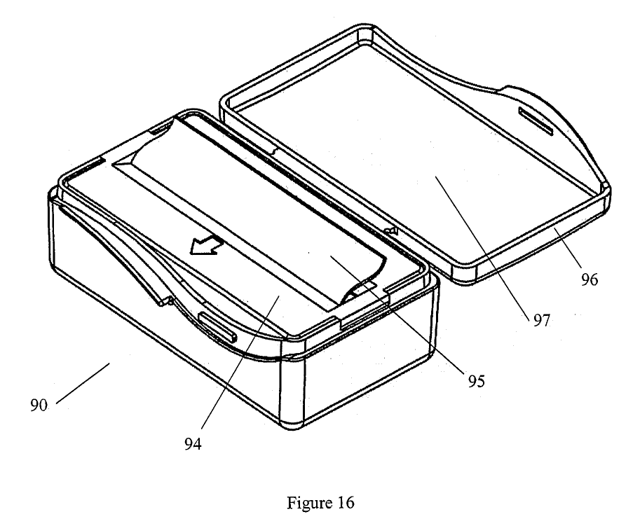

FIG. 15 shows an exploded view of a blood glucose test site cleaning kit according to another embodiment of the present invention.

FIG. 16 shows an assembled view of the blood glucose test site cleaning kit shown in FIG. 15.

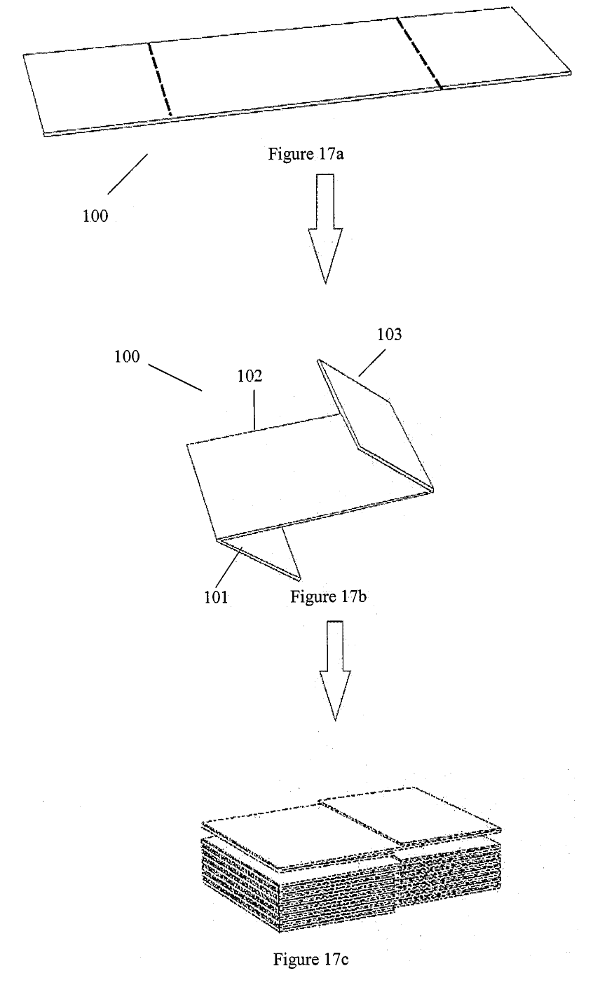

FIGS. 17a, 17b and 17c show a folding arrangement for swabs for use in a blood glucose test site cleaning kit according to the present invention.

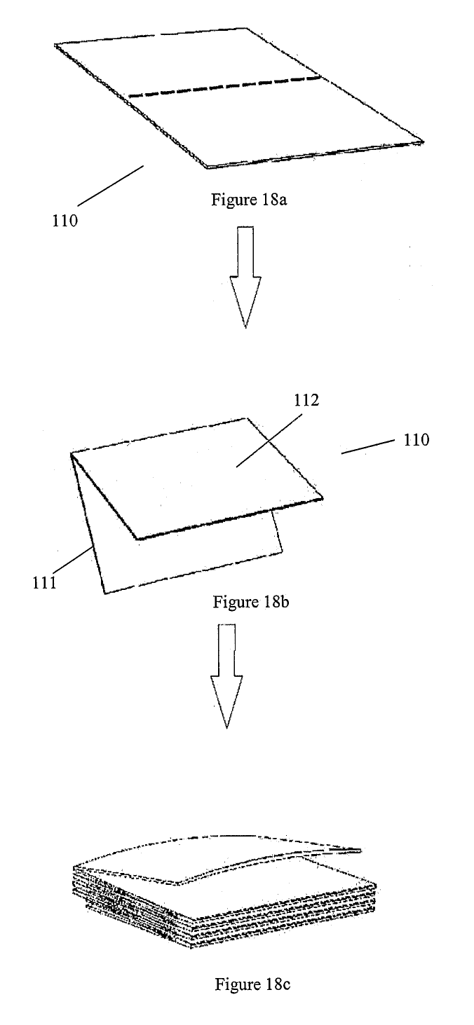

FIGS. 18a, 18b and 18c show another folding configuration for swabs for use in a blood glucose test site cleaning kit according to the present invention.

FIG. 19 shows a blood glucose test site cleaning kit according to another embodiment of the present invention.

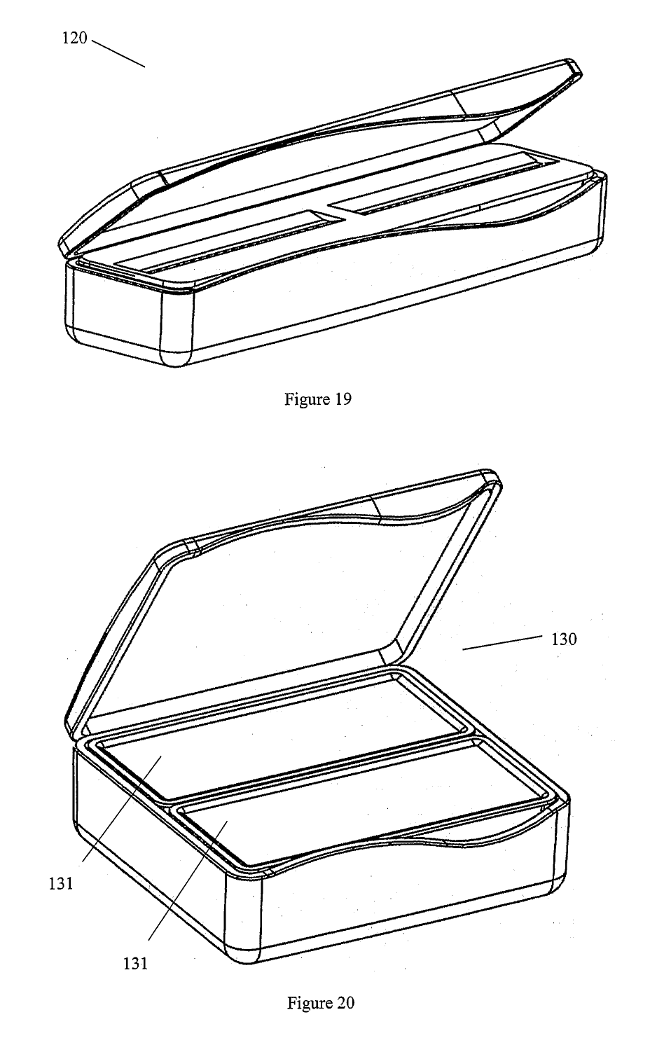

FIG. 20 shows a blood glucose test site cleaning kit according to another embodiment of the present invention.

FIG. 1 shows a container for a blood glucose test site cleaning kit. The container 1 includes a base 2 and a lid 3. The base 2 and the lid 3 are connected by an integral hinge 4. The base is in the form of a walled tray so that swabs (not shown) may be retained in the base 2 in a moisten state.

FIG. 2 shows swabs 5 layered and folded for insertion into the container 1 from FIG. 1. The swabs 5 include a fold back 6 that allows a user to readily remove a single swab from a container. The fold back 6 of adjacent layers are disposed on alternating sides so that efficient packaging of the swabs 5 is provided.

FIGS. 3 and 4 show a blood glucose test site cleaning kit 11. FIG. 3 shows a top view with the lid 13 in a closed condition. Tabs 14 and 15 permit the cleaning kit 11 to be opened to access the swabs. Tab 14 extends from the lid 13 and tab 15 extends from the base 12. Base 12 includes a fastener attachment 16 in the form of a region of loops for a hook and loop fastener adhered to the base 12.

FIGS. 5 and 6 show a blood glucose test site cleaning kit 21 in an open condition. The cleaning kit 21 includes a base 22 and a lid 23. A multiplicity of swabs 5 are layered in the base 22 of the container 21 in a manner similar to that shown in FIG. 2 where a fold back portion 26 extends above the remainder of the swab 25 so that an individual swab can be readily removed.

FIG. 7 shows a blood glucose testing kit 30 which includes a blood glucose test site cleaning kit 31, a blood glucose test meter 32, a lancet 33 and a container of test strips 34. Additional lancets (not shown) for a lancet pen 33 are includes in pouch 35.

FIG. 8 shows the blood glucose test kit of FIG. 7 with the cleaning kit 31 removed from the hook fastener 38 to show how the cleaning kit 31 is retained in the blood glucose test kit 30.

FIG. 9 shows a blood glucose test kit 40 which includes a blood glucose test meter 41, a lancet pen 42, a blood glucose test site cleaning kit 43, three insulin pens 44 and a series of blood glucose test strips 45.

FIG. 10 shows a blood glucose test kit 50 which includes a blood glucose test meter 51, a container of blood glucose test strips 52, a lancet pen 53, a blood glucose test site cleaning kit 54 and additional lancets 55 in a pouch 56.

FIG. 11 shows a blood glucose test cleaning kit 60 in an exploded view. The kit 60 includes a container 61 formed with a recess 62 and a lid 63 joined by an integral hinge 64. Into the recess 62 is a sprung floor 65 having a pair of biasing leaves 66 adapted to urge the base 67 of the sprung floor 65 upwardly to urge swabs contained in the swab cartridge 68 upwardly.

The swab cartridge 68 rests on the sprung floor 65 and is retained within the recess 62. The swab cartridge 68 has a rim 69 which fits within the recess 62. The swab cartridge 68 is sealed for sale and has a removable seal 70 which enables the user to access the swabs. A retainer 71 is mounted over the swab cartridge 68. The retainer 71 includes an elongate, centrally disposed slot through which the swabs may be removed from the swab cartridge 68. The retainer 71 snap fits into the recess 62.

FIG. 12 shows a blood glucose test site cleaning kit 60 as shown in FIG. 11, but in a fully assembled state.

FIGS. 13 and 14 show a blood glucose test site cleaning kit 80 having a recess 81 into which a package of swabs 82 is inserted. The package 82 which has an opening line 83 that permits an enlarged opening in the package 82 to be exposed and through which the swabs may be removed. The swab package 82 is retained within the recess 81 by a retainer 84. The retainer 84 has an enlarged opening 85 to enable the user to readily access the swabs. Preferably the swabs are interleaved as shown in FIGS. 17a, 17b and 17c. Preferably, the container has a footprint of 54 mm×40 mm and contains swabs of 95 mm×35 mm.

FIGS. 15 and 16 show a blood glucose test site cleaning kit 90 having a recess 91 into which a swab cartridge 92 is inserted. A retainer 93 holds the swab cartridge 92 within the recess and aperture 94 permits the user to access the exposed swab 95 once the lid 96 of the kit 90 is open. The lid 96 may include product information 97 which may relate to the swabs, to the use of the swabs, or in fact to the use of a blood glucose test kit. Preferably, the swabs are interleaved as shown in FIGS. 18a, 18b and 18c. Preferably, the container has a footprint of 54 mm×27 mm and contains swabs of 40 mm×40 mm. FIGS. 17a, 17b and 17c show a swab 100 being folded in a “z-fold”. The swab 100 is folded such that a first leaf 101 is doubled over below a middle leaf 102 such that it extends approximately half way below the middle leaf 102. A third leaf 103 is doubled over above the middle leaf 102 such that the distal edges of the first leaf 101 and the third leaf 103 are substantially in alignment. In this way multiple swabs 100 can be stacked in an efficient manner as shown in FIG. 17c.

FIGS. 18a, 18b and 18c show an interleaved configuration of the swabs where the swab 110 is folded in half where a first leaf 111 is disposed immediately below a second leaf 112. The folded swabs 100 are then interleaved so that the upper leaf 112 of a lower folded swab 110 is inserted between the upper leaf 112 and the lower leaf 111 of a swab 110 positioned above the lower swab. The interleaved folded swabs can be seen in FIG. 18c.

FIG. 19 shows a blood glucose test site cleaning kit 120 having dual recesses for storing two sets of swabs. The recesses are aligned in side to side arrangement.

FIG. 20 shows a blood glucose test site cleaning kit 130 which has two recesses for receiving test swabs. The recesses 131 are disposed front to back whereby a relatively square shaped kit 130 is provided.

Persons skilled in the art will appreciate that the invention described above may be subject to improvements and modifications that will be apparent without departing from the spirit and scope of the invention described herein.

What is claimed is:

1. A diabetes blood glucose test site cleaning kit comprising a container housing a multiplicity of swabs wherein the swabs are layered within the container to facilitate one by one removal and wherein the swabs are moistened with an aqueous cleansing solution, the container being dimensioned such that the cleaning kit readily fits into a blood glucose meter wallet.

2. The diabetes blood glucose test site cleaning kit according to claim 1 wherein the container has a foot print of less than about 55 mm by about 90 mm.

3. The diabetes blood glucose test site cleaning kit according to claim 1 wherein the container has a foot print of about 54 mm by 27 mm.

4. The diabetes blood glucose test site cleaning kit according to claim 3 wherein the swab is 40 mm×40 mm.

5. The diabetes blood glucose test site cleaning kit according to claim 1 wherein the container has a footprint of 54 mm×40 mm.

6. The diabetes blood glucose test site cleaning kit according to claim 5 wherein the swab is 95 mm×35 mm.

7. The diabetes blood glucose test site cleaning kit according to claim 1 wherein a thickness of the container is in the range of from 10 to 20 mm.

8. The diabetes blood glucose test site cleaning kit according to claim 1 wherein the container is configured to receive refills of the swabs.

9. The diabetes blood glucose test site cleaning kit according to claim 8 wherein the refills are provided in the form of sealed bags and sealed cartridges.

10. The diabetes blood glucose test site cleaning kit according to claim 1 wherein the container has a retaining member that holds a refill in the container during use and that can be removed for replacement of the refill.

11. The diabetes blood glucose test site cleaning kit according to claim 1 wherein the container includes a sprung floor insert that elevates the swabs to the top of the container for ready access to the swabs.

12. The diabetes blood glucose test site cleaning kit according to claim 11 wherein the sprung floor includes a support having integrally moulded, downwardly extending leaves that biases the support to a raised condition.

13. The diabetes blood glucose test site cleaning kit according to claim 1 wherein the container is an injection moulded plastics container.

14. The diabetes blood glucose test site cleaning kit according to claim 1 wherein the container has an integral hinge such that the swabs can be readily accessed by the user and the container resealed for subsequent storage and use.

15. The diabetes blood glucose test site cleaning kit according to claim 1 wherein the swabs may be made from a textile fabric, an absorbent plastic or a paper based material.

16. The diabetes blood glucose test site cleaning kit according to claim 1 wherein the swabs are simply layered in the container.

17. The diabetes blood glucose test site cleaning kit according to claim 1 wherein the swabs are interlayered, whereby the swabs are folded such that each swab extends partway into an adjacent layer leaving an end that may be readily accessed by the user for the removal of the next swab.

18. The diabetes blood glucose test site cleaning kit according to claim 17 wherein the swabs are interlayered using a “z-fold”.

19. The diabetes blood glucose test site cleaning kit according to claim 1 wherein the swabs are interleaved whereby the withdrawal one swab positions the next for withdrawal through a centrally disposed slot in the container.

20. The diabetes blood glucose test site cleaning kit according to claim 1 wherein the cleaning solution includes surfactants for assisting in the removal of sugars and other contaminants from the test site.

21. The diabetes blood glucose test site cleaning kit according to claim 1 wherein the cleansing solution includes preservatives.

22. The diabetes blood glucose test site cleaning kit according to claim 1 wherein the diabetes blood glucose test site cleaning kit includes a display of selected information about site preparation for administering a blood glucose test or other aspects of diabetes management.

23. The diabetes blood glucose test site cleaning kit according to claim 1 wherein the diabetes blood glucose test site cleaning kit includes a fastener on the container for fixing the container to the wallet.
